(19) United States
(12) Patent Application Publication
(10) Pub. No.: US 2023/0133732 A1
(43) Pub. Date: May 4, 2023

(54) INFORMATION PRESENTING APPARATUS, INFORMATION PRESENTING METHOD AND PROGRAM

(71) Applicant: NIPPON TELEGRAPH AND TELEPHONE CORPORATION, Tokyo (JP)

(72) Inventors: Tomu TOMINAGA, Tokyo (JP); Takeshi KURASHIMA, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/917,516

(22) PCT Filed: Apr. 13, 2020
(86) PCT No.: PCT/JP2020/016265

§ 371 Date: Oct. 6, 2022

(51) Int. Cl. A61B 5/00; A61B 5/11

(52) U.S. Cl. CPC A61B 5/7275; A61B 5/1118

(57) ABSTRACT

An information presentation device of the present invention presents information regarding a risk to a user. The information presentation device includes a processor and a memory storing program instructions that cause the processor to acquire an objective risk value regarding the user, acquire a subjective risk value regarding the user, and present the information regarding the risk to the user based on the subjective risk value and the objective risk value.

### TECHNICAL FIELD

The present invention relates to a technique for presenting information related to a risk such as a health risk to a user based on evaluation values.

### BACKGROUND ART

In recent years, the relationship that activity history data has with objective evaluation values and subjective evaluation values regarding an individual's health risks has been attracting attention in the design of mobile computer technology for utilizing data regarding people's daily activity in order to improve their health condition. Hereinafter, activity history data is simply referred to as activity logs.

NPL 1 and 2 disclose examples of conventional techniques for utilizing such data. NPL 1 and 2 disclose studies on the influence that subjective evaluation values of a person's health risk have on activity logs. Also, NPL 2 discloses a technique for predicting an objective evaluation value based on activity logs, a technique for revealing a correlation between subjective evaluation values and objective evaluation values, and the like.

### CITATION LIST

### Non Patent Literature

[NPL 1] Hershman, Steven G., et al. “Physical activity, sleep and cardiovascular health data for 50,000 individuals from the MyHeart Counts Study.” Scientific data 6.1 (2019): 24.

[NPL 2] McConnell, Michael V., et al. “Feasibility of obtaining measures of lifestyle from a smartphone app: the MyHeart Counts Cardiovascular Health Study.” JAMA cardiology 2.1 (2017): 67-76.

### SUMMARY OF THE INVENTION

### Technical Problem

As described above, there are conventional techniques for analyzing the influence that subjective evaluation values have on activity logs and for analyzing the tendency for similarity between a subjective evaluation value and an objective evaluation value. There are also many conventional techniques for presenting recommendations and alerts to users based on objective evaluation values of health risks.

However, if a user is subjectively aware of a risk, it is not very effective to present a recommendation or an alert simply because an objective health risk exists. In other words, consideration needs to be given to both a subjective evaluation value and an objective evaluation value when presenting information regarding a health risk to a user, but there is no conventional technology that achieves this.

The present invention has been made in view of the above points, and an object of the present invention is to provide a technique that gives consideration to both a subjective evaluation value and an objective evaluation value, detects a discrepancy between the subjective evaluation value and the objective evaluation value, and determines an interaction to be taken with a user (e.g., presentation of information for addressing a health risk or marketing analysis target selection).

### Means for Solving the Problem

One aspect of the disclosed technology is an information presentation device that presents information regarding a risk to a user, including:

an objective risk value acquisition unit configured to acquire an objective risk value regarding the user;

a subjective risk value acquisition unit configured to acquire a subjective risk value regarding the user; and

a presentation unit configured to present the information regarding the risk to the user based on the subjective risk value and the objective risk value.

### Effects of the Invention

According to the disclosed technology, it is possible to provide a technique that gives consideration to both a subjective evaluation value and an objective evaluation value, detects a discrepancy between the subjective evaluation value and the objective evaluation value, and determines an interaction to be taken with a user.

### BRIEF DESCRIPTION OF DRAWINGS

FIG. 1 is a configuration diagram of a device according to a first embodiment of the present invention.

FIG. 2 is a flowchart showing operations of an activity estimation device.

FIG. 3 is a diagram showing an example of subjective evaluation values regarding heart age stored in a subjective evaluation value storage unit 1.

FIG. 4 is a diagram showing an example of objective evaluation values regarding heart age stored in a subjective evaluation value storage unit 2.

FIG. 5 is a diagram showing an example of activity logs stored in an activity log storage unit 6.

FIG. 6 is a diagram showing an example of health risk discrepancy values regarding heart age stored in a health risk discrepancy value storage unit 5.

FIG. 7 is a diagram showing an example of activity log correlation coefficients.

FIG. 8 is a diagram showing an example of activity logs stored in a selected variable storage unit 9.

FIG. 9 is a diagram showing an example of measurement results displayed by a result display unit 11.

FIG. 10 is a configuration diagram of a device according to a second embodiment of the present invention.

FIG. 11 is a diagram showing an example of a device hardware configuration.

### DESCRIPTION OF EMBODIMENTS

Hereinafter, embodiments of the present invention (present embodiments) will be described with reference to the drawings. The embodiments described below are merely examples, and the embodiments to which the present invention is applicable are of course not limited to the following embodiments.

Hereinafter, a first embodiment and a second embodiment will be described. In the following embodiments, a health risk is applied as an example of the risk, but a health risk is merely one example of a risk. The present invention is not limited to being applied to a health risk, and is applicable to various risks.

The term “objective evaluation value” that appears in the following description means a value obtained by quantifying how high a health risk is based on multiple pieces of biological information (e.g., weight, age, blood pressure), and is a concept that includes a numerical value of stroke risk, heart age, or the like. On the other hand, the term “subjective evaluation value” means a value that quantifies how high a self-recognized health risk is, and mainly refers to a value measured by a questionnaire or the like. Note that the objective evaluation value may also be referred to as an objective risk value, and the subjective evaluation value may also be referred to as a subjective risk value. Also, the subjective evaluation value may be an actual numerical value if it is a parameter related to a health risk that the user is aware of. Typical examples of actual numerical values include the blood pressure and the heart rate according to the user (not measured values).

Also, the activity log in the following description is a time-continuous record of activity in daily life, and is a concept that includes logs such as a record of steps or burned calories measured/calculated by GPS or an accelerometer, or a record of meals or sleep obtained by a self-reported questionnaire.

### First Embodiment

First, a first embodiment will be described. In the aforementioned conventional techniques, correlations between subjective evaluation values and objective evaluation values of a health risk are examined, and the tendency of similarity between these two types of evaluation values is checked for the subject as a whole.

However, conventional technology has not been able to (1) perform an individual-level comparison of discrepancy between a subjective evaluation value and an objective evaluation value, (2) reveal the relationship between activity logs and the levels of a subjective evaluation value and an objective evaluation value, and (3) reveal a dominant activity log with respect to the levels of a subjective evaluation value and an objective evaluation value.

Accordingly, with conventional technology, it is not possible to give consideration to both a subjective evaluation value and an objective evaluation value, detect a discrepancy between the subjective evaluation value and the objective evaluation value, and determine an interaction that is to be taken with a user.

In view of the above points, in the first embodiment, an activity estimation device 100 specifies a discrepancy between a subjective evaluation value and an objective evaluation value of an individual's health risk as well as a dominant activity that is a cause for the discrepancy, and makes it possible for health improvement assistance to be given for an appropriate activity in accordance with a user's level of understanding of their own health risk.

<Overview>

The following describes an overview of operations of the activity estimation device 100 in the present embodiment.

The activity estimation device 100 first quantifies the extent of discrepancy between subjective evaluation values and objective evaluation values of an individual's health risk (hereinafter referred to as the health risk discrepancy value). At this time, in order to be able to handle various evaluation values, the subjective evaluation values and the objective evaluation values may be normalized in advance.

Next, the activity estimation device 100 performs regression analysis using the health risk discrepancy values as the response variable and using activity logs as the explanatory variable, and measures the effect that the activity logs have on the health risk discrepancy values. At this time, in order to be able to handle various activity logs, all of the activity log data is normalized in advance.

Also, in order to avoid multicollinearity, the explanatory variables that are to be used for analysis are selected according to certain criteria (e.g., the absolute value of the correlation coefficient is 0.8 or more, but the criteria for the correlation coefficient may be designed in accordance with the field of application). This normalization and variable selection eliminates dependency between activity logs and reveals a more accurate relationship between health risk discrepancy values and activity logs. This also contributes to the more accurate specification of dominant activity logs for a health risk discrepancy value.

The activity estimation device 100 specifies dominant activities for a health risk discrepancy value based on the effect measurement results. The specified activities are presented to the user.

With the technology according to the present embodiment, it is possible to reveal a robust relationship between an individual health risk discrepancy value and activity logs of an individual, and to specify dominant activity logs for the health risk discrepancy value with high accuracy. Accordingly, it is possible to provide health improvement assistance for an appropriate activity in accordance with an individual's level of understanding of their own health risk.

Hereinafter, the configuration and operation of the activity estimation device 100 will be described in detail.

<Configuration of Activity Estimation Device 100>

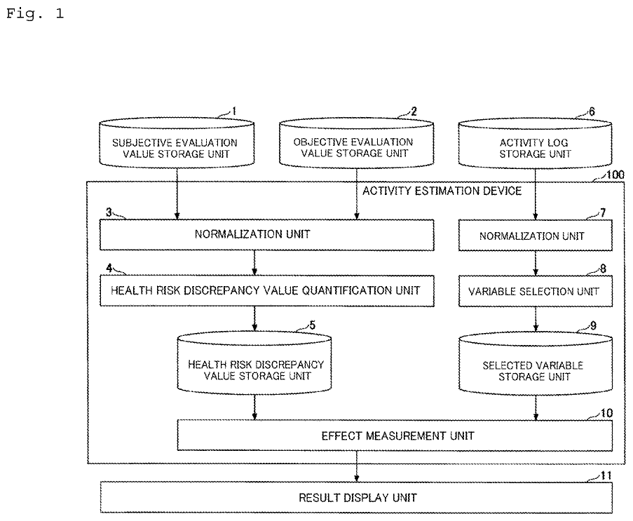

FIG. 1 shows an example of the configuration of the activity estimation device 100. As shown in FIG. 1, the activity estimation device 100 includes a normalization unit 3, a health risk discrepancy value quantification unit 4, a health risk discrepancy value storage unit 5, a normalization unit 7, a variable selection unit 8, a selected variable storage unit 9, and an effect measurement unit 10.

As shown in FIG. 1, the activity estimation device 100 is connected to a subjective evaluation value storage unit 1, an objective evaluation value storage unit 2, an activity log storage unit 6, and a result display unit 11. Also, any or all of the subjective evaluation value storage unit 1, the objective evaluation value storage unit 2, the activity log storage unit 6, and the result display unit 11 may be provided in the activity estimation device 100.

Note that the activity estimation device 100 including the result display unit 11 may be referred to as an “information presentation device”. Also, in the normalization unit 3, the function of normalizing data acquired from the objective evaluation value storage unit 2 may be referred to as an “objective risk value acquisition unit”. In the normalization unit 3, the function of normalizing data acquired from the subjective evaluation value storage unit 1 may be referred to as a “subjective risk value acquisition unit”. Also, the normalization unit 7 may be referred to as an “activity log acquisition unit”. Moreover, the effect measurement unit 10 and the result display unit 11 may be collectively referred to as a “presentation unit”.

<Operation of Activity Estimation Device 100>

In the present embodiment, heart ages, which serve as indicators of a health risk, are acquired from users and stored in the subjective evaluation value storage unit 1 and the objective evaluation value storage unit 2 as subjective evaluation values and objective evaluation values. It is also envisioned that a step count, a walking distance, a cycling distance, and an altitude in daily life are observed as activity logs for each user, and the observation results are stored in the activity log storage unit 6. It is assumed here that there are N users.

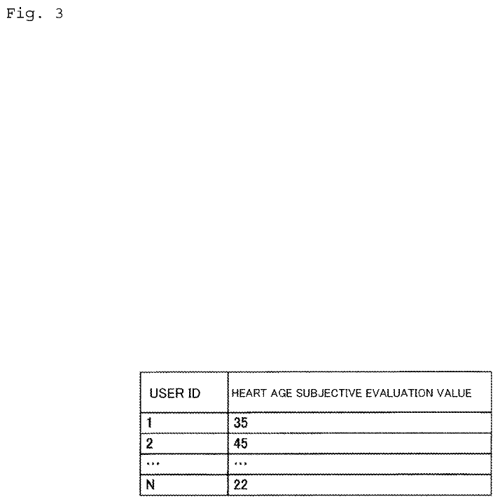

FIG. 3 shows an example of data stored in the subjective evaluation value storage unit 1. In FIG. 3, it is shown that the subjective evaluation value of the heart age of the user with user ID=2 is 45, for example.

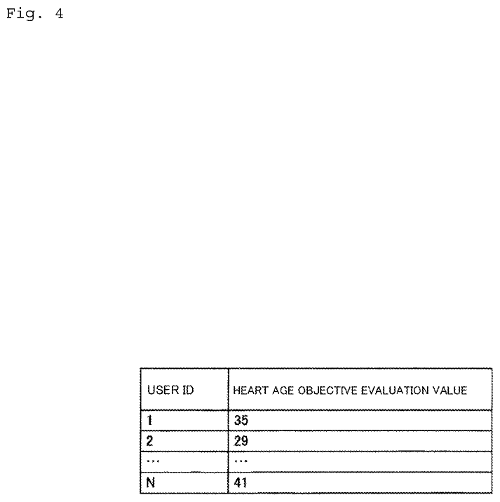

FIG. 4 shows an example of data stored in the objective evaluation value storage unit 2. In FIG. 4, it is shown that the objective evaluation value of the heart age of the user with user ID=2 is 29, for example.

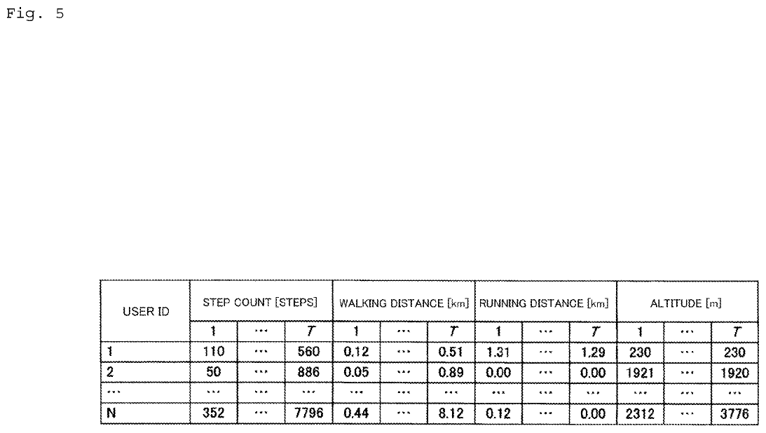

FIG. 5 shows an example of data stored in the activity log storage unit 6. In FIG. 5, 1 to T indicate time stamps. As shown in FIG. 5, a value is stored together with a time stamp for each user and each type of activity in the activity log.

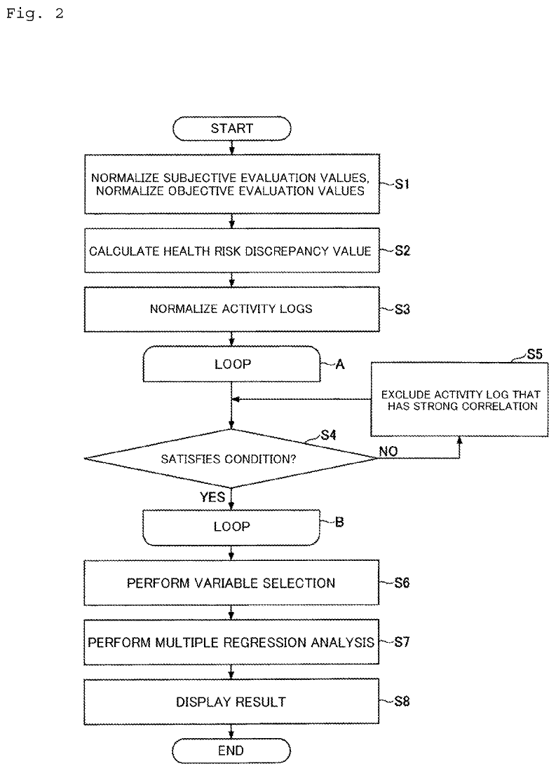

Hereinafter, an example of operation of the activity estimation device 100 will be described in detail in accordance with the procedure shown in the flowchart of FIG. 2.

<Step S1>

The subjective evaluation value storage unit 1 and the objective evaluation value storage unit 2 transmit data stored therein to the normalization unit 3 in accordance with a request from the activity estimation device 100. In step S1, the normalization unit 3 performs normalization on the evaluation values received from the subjective evaluation value storage unit 1 and the objective evaluation value storage unit 2 for conversion to scale that enables comparison even if the units of the evaluation values are different.

In the present embodiment, the normalization unit 3 subjects the evaluation values to normalization with an average of 0 and a standard deviation of 1 (affine transformation). However, this is one example, and the normalization unit 3 may perform linear conversion with a root mean square of 1 (proportional conversion), normalization with a maximum value of 1 and minimum value of 0, or another type of normalization.

The subjective evaluation values and the objective evaluation values that were normalized by the normalization unit 3 will be respectively denoted by “˜sᵢ” and “˜oᵢ”. Although the notation “˜” in “˜sᵢ” is intended to be placed above “s”, it is placed before “s” for convenience of description in the text of the specification. The same applies to “˜oᵢ” and the like hereinafter. Note that i=1, . . . , N. In the present embodiment, it is assumed that for both the subjective evaluation value and the objective evaluation value, the larger the value is, the higher the health risk is.

The normalization unit 3 transmits the normalized evaluation values to the health risk discrepancy value quantification unit 4.

<Step S2>

In step S2, the health risk discrepancy value quantification unit 4 calculates a health risk discrepancy value based on a normalized subjective evaluation value and a normalized objective evaluation value. Letting the health risk discrepancy value be yᵢ, and the function for calculating the health risk discrepancy value be f, then yᵢ=f(˜sᵢ, ˜oᵢ). In the present embodiment, the health risk discrepancy value quantification unit 4 calculates the difference between a normalized subjective evaluation value and a normalized objective evaluation value (˜sᵢ−˜oᵢ) as the health risk discrepancy value. In other words, the value obtained by subtracting the normalized objective evaluation value from the normalized subjective evaluation value is used as the health risk discrepancy value.

Note that calculating the difference between the subjective evaluation value and the objective evaluation value as the health risk discrepancy value is merely one example, and the health risk discrepancy value may be calculated by any method that can numerically express a discrepancy between the normalized subjective evaluation value and the normalized objective evaluation value. For example, relative error (1−(˜oᵢ/˜sᵢ)) may be used as the health risk discrepancy value, or a category value representing a magnitude relationship (if (˜sᵢ)>(˜oᵢ), then 1, else 0) may be used as the health risk discrepancy value. For example, if ˜sᵢ is 2 and ˜oᵢ is 1, then the category value is 1.

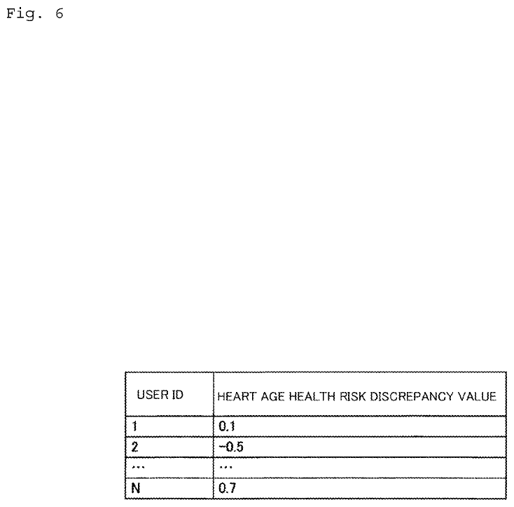

The health risk discrepancy value quantification unit 4 transmits the calculated health risk discrepancy value to the health risk discrepancy value storage unit 5. FIG. 6 shows an example of data stored in the health risk discrepancy value storage unit 5. As shown in FIG. 6, a health risk discrepancy value is stored for each user ID.

<Step S3>

The activity log storage unit 6 stores activity logs. As shown in FIG. 5, each activity log is stored as time-series data in the activity log storage unit 6. In accordance with a request from the activity estimation device 100, the activity log storage unit 6 reads one or more types of activity logs, concatenates the time-series data, and transmits the data to the normalization unit 7 of the activity estimation device 100.

For example, let time-series data (t=1, 2, . . . , T) for an activity b∈B={step, run, walk, height} for a user uᵢ be a vector dᵢ^{b} that contains pieces of log data lᵢ^{b,t} of the activity b in chronological order. In other words, dᵢ^{b}=(lᵢ^{b,l}, lᵢ^{b,2}, . . . , lᵢ^{b,T}). At this time, let the data xᵢ of each user transmitted by the activity log storage unit 6 to the normalization unit 7 of the activity estimation device 100 be xᵢ=(dᵢ^{step}, dᵢ^{run}, dᵢ^{walk}, dᵢ^{height})^{T}.

The normalization unit 7 normalizes the received activity log data by affine transformation. The thus normalized activity log data is then sent to the variable selection unit 8. Let the normalized activity log data of each user be zᵢ=(dᵢ^{-b1}, . . . , dᵢ^{-b|B|})^{T}. Note that the superscript “˜b1, . . . , ˜b|B|” at the upper right is intended to be ˜b₁, . . . , ˜b_{|B|}.

<Steps S4, S5>

Step S4 (S5) is executed for each activity combination (bₘ,b_{m′}) (loop from A to B in FIG. 2).

In step S4, the variable selection unit 8 excludes activity logs that show a strong correlation with each other, based on the activity logs that were normalized by the normalization unit 7. The variable selection unit 8 determines the strength of correlation between activity logs based on a correlation coefficient obtained by correlation analysis.

Let γ_{b,b′} be the correlation coefficient between two activities b and b′. When |γ_{b,b′}|>γ′ (where 0<γ′<1) is satisfied for a certain threshold value γ′, the variable selection unit 8 excludes either one of the activity logs b and b′.

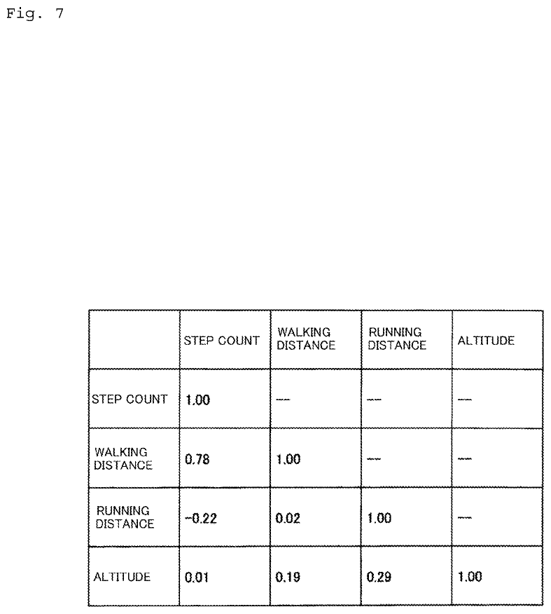

In the flowchart shown as an example in FIG. 2, in step S4, the variable selection unit 8 makes a condition determination using the condition |γ_{b,b′}|<γ′, and if Yes, processing moves to the next loop, and if No, either one of the activity logs b and b′ is excluded, and processing moves to the next loop. For example, assume that the results shown in FIG. 7 are obtained when the variable selection unit 8 checks the correlation coefficients of the activity logs.

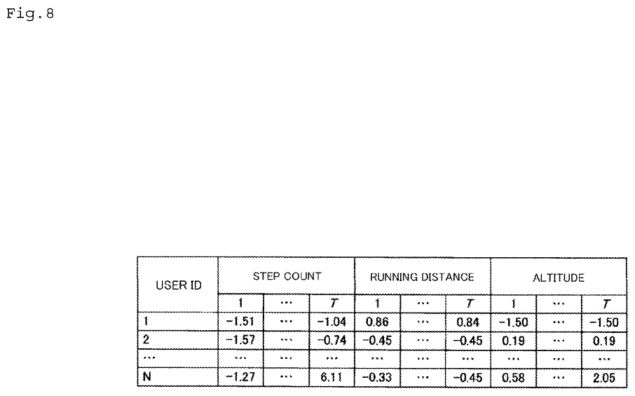

Here, it is assumed that γ′=0.40 is set as the threshold value. At this time, the magnitude of the correlation coefficient between the step count and the walking distance exceeds the threshold value, and therefore the walking distance is excluded from the activity logs. As a result, the variable selection unit 8 transmits the activity log regarding the step count, the running distance, and the altitude to the selected variable storage unit 9. FIG. 8 shows an example of the data stored in the selected variable storage unit 9.

<Step S6>

The variable selection unit 8 sets zᵢ′ as the selected activity log of the user uᵢ for each user after excluding one of two activity logs that have a high correlation coefficient.

<Step S7>

The health risk discrepancy value storage unit 5 (e.g., FIG. 6) and the selected variable storage unit 9 (FIG. 8) transmit data stored therein to the effect measurement unit 10.

The effect measurement unit 10 receives the health risk discrepancy values from the health risk discrepancy value storage unit 5, receives the selected activity logs from the selected variable storage unit 9, performs regression analysis using the health risk discrepancy values as the response variable and using the selected activity log as the explanatory variable, and measures the effect that the activity logs included in the selected activity logs have on the discrepancy between the subjective evaluation values and the objective evaluation values of a health risk.

If a method of expressing the health risk discrepancy value with a category value is adopted in the health risk discrepancy value quantification unit 4, the effect measurement unit 10 performs logistic regression analysis. Also, if a method of expression with a numerical scale such as a difference or relative error is adopted in the health risk discrepancy value quantification unit 4, the effect measurement unit 10 performs multiple regression analysis.

The regression analysis performed by the effect measurement unit 10 corresponds to solving the optimization problem shown in Expression 1 shown below.

$$\begin{matrix}[ Math . 1 ] & \\ {argmin}_{β} \{ {\sum }_{i} {( {y}_{i} - ( {β}^{T} \cdot {z}_{i}^{′} ) )}^{2} \} & Exp . 1\end{matrix}$$

In Expression 1, β is a coefficient vector corresponding to each activity log in zᵢ′. For example, when zᵢ′=(˜d^{step}, ˜d^{run}, ˜d^{height})^{T}, then β=(βₛₜₑₚ, βᵣᵤₙ, β_{height})^{T}. The elements (coefficients) in the coefficient vector show the effect that the corresponding activity log has on the health risk discrepancy value. For example, assume that the following effect β is obtained as a result of solving the optimization problem shown in Expression 1.

β=(βₛₜₑₚ,βᵣᵤₙ,β_{height})^{T}−(−0.22,1.56,0.19)^{T}

Here, βₛₜₑₚ, βᵣᵤₙ, and β_{height} correspond to the coefficients related to the step count, the running distance, and the altitude, respectively. Assume that statistical significance is confirmed for each coefficient at the significance level p=0.01. These effect measurement results are transmitted to the result display unit 11.

<Step S8>

The result display unit 11 receives the measurement results obtained by the effect measurement unit 10 from the activity estimation device 100 and displays the measurement results. The result display unit 11 displays the activity log names in correspondence with effects in descending order of the absolute value of the measured effect, for example. Here, “display” is a concept that includes transmission to an external device such as printing on a display or by a printer, and is realized by an output device and driver software for the same.

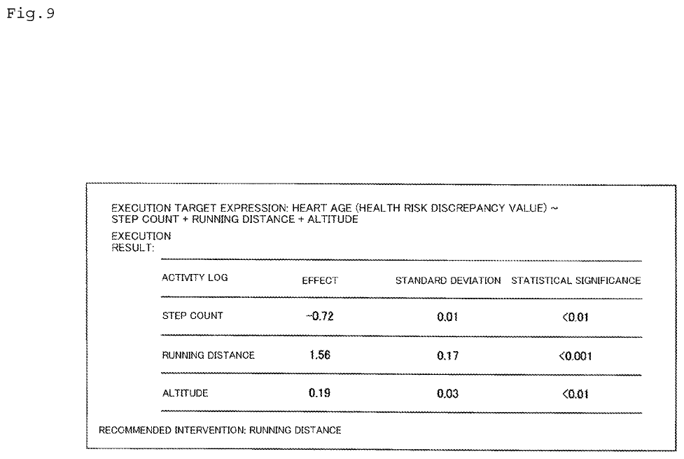

For example, the result display unit 11 displays the measurement results received from the effect measurement unit 10 as shown in FIG. 9.

In FIG. 9, the “execution target expression” shows the relationship between the response variable (left side) and the explanatory variable (right side) in the multiple regression analysis. “Execution result” displays the effect of each activity log, which is the explanatory variable. Also, the standard deviation and statistical significance of the effect of each activity log are also displayed.

It can be interpreted from the results illustrated in FIG. 9 that (1) the running distance has the greatest effect on the health risk discrepancy value of heart age, (2) as the step count increases, the health risk discrepancy value of heart age decreases (takes a negative value), and (3) as the running distance and altitude increase, the health risk discrepancy value of heart age increases (takes a positive value).

In other words, based on the results shown in FIG. 9, it can be understood that increasing the running distance effectively leads to a positive change in the health risk discrepancy value. Changing the health risk discrepancy value (“subjective evaluation value—objective evaluation value”) in the positive direction is a concept that includes lowering the objective evaluation value (objective health risk), and this can be said to mean that the user is healthier. In other words, it can be said that increasing the running distance is an important activity to be taken for the user to become healthier.

Based on these measurement results and interpretations, the “recommended intervention” in FIG. 9 displays the names of the most important activity logs that contribute to improving the user's health condition.

As described above, according to the first embodiment, activity logs that are dominant for the health risk discrepancy value can be specified with high accuracy. In other words, according to the first embodiment, the cause of the health risk discrepancy and the effect thereof can be specified, thus making it possible to determine an intervention strategy for inspiring a person to change their activity in order to suppress risk.

### Second Embodiment

Next, a second embodiment will be described.

<Overview>

As described in NPL 2, there are many techniques for objectively evaluating a risk in a person, and similarly, there are many techniques for presenting recommendations and alerts to a user based on objective evaluation values.

However, if a user is subjectively aware of a risk, it is not very effective to present a recommendation or an alert simply because an objective risk exists. A user who should receive a recommendation or an alert is a user for which the difference between a subjective risk evaluation value and an objective risk evaluation value has a large absolute value. In other words, a user who should receive a recommendation or an alert is a user who is not subjectively aware of a risk but objectively has a risk, or a user who is subjectively aware of a risk but does not objectively have a risk.

Specifically, in the case of “a user who is not subjectively aware of a risk but objectively has a risk”, if the user objectively has a weight-related risk but does not subjectively recognize the risk for example, an alert regarding adult disease is presented, or a recommendation is presented for an amount by which consumption of food/drink is to be decreased or an amount by which exercise is to be increased.

In the case of “a user who is subjectively aware of a risk but does not objectively have a risk”, a recommendation is presented regarding an amount of increase in consumption of food/drink that is envisioned to be acceptable with respect to a risk regarding adult disease.

<Device Configuration and Operation>

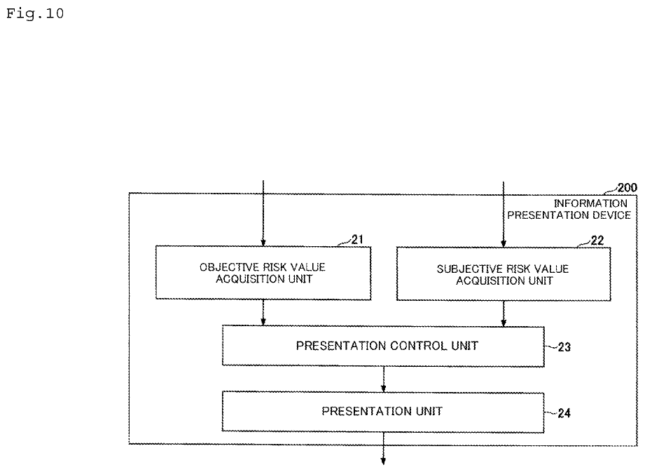

FIG. 10 shows an example of the configuration of an information presentation device 200 that presents risk-related information such as that described above to a user. As shown in FIG. 10, the information presentation device 200 includes an objective risk value acquisition unit 21, a subjective risk value acquisition unit 22, a presentation control unit 23, and a presentation unit 24. Note that the presentation control unit 23 and the presentation unit 24 may be collectively referred to as a “presentation unit”. The operation of the information presentation device 200 will be described below. Hereinafter, the user to whom information is presented will be referred to as the target user.

The objective risk value acquisition unit 21 acquires objective risk values regarding the target user, and the subjective risk value acquisition unit 22 acquires subjective risk values regarding the target user. The objective risk values and the subjective risk values are, for example, evaluation values related to a health risk similar to the objective evaluation values and the subjective evaluation values described in the first embodiment. The objective risk values and the subjective risk values regarding the target user may be a value indicating a physical risk or a mental risk regarding the target user.

Also, the objective risk values and the subjective risk values that are acquired may be normalized information similarly to the objective evaluation values and the subjective evaluation values described in the first embodiment.

The objective risk values and the subjective risk values may be acquired by input from the target user, or may be acquired from a database or the like that stores such information in advance.

The presentation control unit 23 receives the objective risk values of the target user from the objective risk value acquisition unit 21, and receives the subjective risk values of the target user from the subjective risk value acquisition unit 22.

The presentation control unit 23 determines whether or not to present risk-related information to the target user based on the subjective risk values and the objective risk values, and in the case of presentation, determines the information that is to be presented. For example, the presentation control unit 23 determines that risk-related information is to be presented to the target user only if the absolute value of the difference between an objective risk value and a subjective risk value is greater than a predetermined threshold value.

Here, it is assumed that for both the objective risk values and the subjective risk values, the larger the values are, the greater the risk is. In the case where it was determined that risk-related information is to be presented to the target user, if “objective risk value <subjective risk value” holds true, the presentation control unit 23 determines that the target user is a “user who is subjectively aware of a risk but does not objectively have a risk”. The presentation control unit 23 then determines a recommendation is to be given regarding an amount by which consumption of food/drink may be increased, as previously described. As for the content of the recommendation, predetermined content may be used, or a model trained by machine learning may be used to determine the content in accordance with the magnitude of “subjective risk value—objective risk value”.

In the case where it was determined that risk-related information is to be presented to the target user, if “objective risk value >subjective risk value” holds true, the presentation control unit 23 determines that the target user is a “user who is not subjectively aware of a risk but objectively has a risk”. The presentation control unit 23 then determines that an alert regarding adult disease is to be presented, or a recommendation is to be presented for an amount by which consumption of food/drink is to be decreased or an amount by which exercise is to be increased, as previously described. As for the content of the alert or the recommendation, predetermined content may be used, or a model trained by machine learning may be used to determine the content in accordance with the magnitude of “subjective risk value—objective risk value”.

If it was determined that risk-related information is to be presented to the target user, the presentation control unit 23 transmits presentation information to the presentation unit 24, and the presentation unit 24 presents the information (above-described recommendation or the like) to the target user. Here, “presentation” may include display on a display or the transmission of information to a terminal held by the target user.

### Hardware Configuration Example

Both the activity estimation device 100 and the information presentation device 200 of the embodiments can be realized by, for example, causing a computer to execute a program describing the processing content described in the embodiments. Note that the “computer” may be a physical machine or a virtual machine in the cloud. When using a virtual machine, the “hardware” described here is virtual hardware.

The aforementioned program can be recorded on a computer-readable recording medium (portable memory, etc.), stored, and distributed. It is also possible to provide the above program through a network such as the Internet or e-mail.

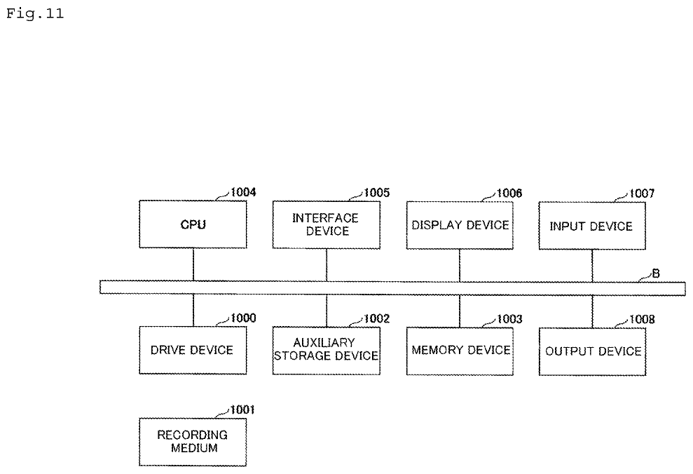

FIG. 11 is a diagram showing an example of the hardware configuration of the computer. The computer shown in FIG. 11 has a drive device 1000, an auxiliary storage device 1002, a memory device 1003, a CPU 1004, an interface device 1005, a display device 1006, an input device 1007, an output device 1008, and the like, which are connected to each other by a bus BS.

The program that realizes the processing in the computer is provided via, for example, a recording medium 1001 such as a CD-ROM or a memory card. When the recording medium 1001 storing the program is set in the drive device 1000, the program is installed from the recording medium 1001 to the auxiliary storage device 1002 via the drive device 1000. However, the program is not necessarily required to be installed from the recording medium 1001, and may be downloaded from another computer via a network. The auxiliary storage device 1002 stores the installed program as well as necessary files and data.

When an instruction to start the program is received, the memory device 1003 reads the program from the auxiliary storage device 1002 and stores it. The CPU 1004 realizes the functions pertaining to the device in accordance with the program stored in the memory device 1003. The interface device 1005 is used as an interface for connecting to a network. The display device 1006 displays a programmatic GUI (Graphical User Interface) and the like. The input device 1007 is constituted by a keyboard, a mouse, buttons, a touch panel, or the like, and is used for inputting various operation instructions. The output device 1008 outputs computation results.

### Effects of the Embodiments

As described above, according to the embodiments of the present invention, there is provided a technique capable of presenting risk-related information to a user based on both a subjective evaluation value and an objective evaluation value.

### Summary of the Embodiments

At least an information presentation device, an information presentation method, and a program described the following items are described in the present specification.

### (Item 1)

An information presentation device that presents information regarding a risk to a user, including:

an objective risk value acquisition unit configured to acquire an objective risk value regarding the user;

a subjective risk value acquisition unit configured to acquire a subjective risk value regarding the user; and

a presentation unit configured to present the information regarding the risk to the user based on the subjective risk value and the objective risk value.

### (Item 2)

The information presentation device according to item 1,

wherein the risk is a physical risk regarding the user or a mental risk regarding the user.

### (Item 3)

The information presentation device according to item 1 or 2,

wherein the presentation unit presents the information regarding the risk to the user only if a difference between the objective risk value and the subjective risk value is greater than a predetermined threshold value.

### (Item 4)

An information presentation device that presents information regarding an activity to be taken with respect to a risk pertaining to health of a user, the information presentation device including:

an objective risk value acquisition unit configured to acquire objective risk values that indicate an objective health-related risk regarding a plurality of users;

a subjective risk value acquisition unit configured to acquire subjective risk values regarding the plurality of users, the subjective risk values indicating a risk subjectively recognized by the plurality of users;

an activity log acquisition unit configured to acquire activity logs regarding the plurality of users; and

a presentation unit configured to, for each of the plurality of users, present information regarding an activity to be taken with respect to a risk, based on a risk discrepancy value indicating a discrepancy between the subjective risk value and the objective risk value regarding the user and based on the activity log regarding the user.

### (Item 5)

The information presentation device according to item 4,

wherein the presentation unit specifies activity logs that are dominant with respect to the risk discrepancy value by performing regression analysis on the risk discrepancy value and the activity logs, and presents a name of the activity log as the information regarding an activity to be taken with respect to the risk.

### (Item 6)

An information presentation method executed by an information presentation device that presents information regarding a risk to a user, the information presentation method including:

an objective risk value acquiring step of acquiring an objective risk value regarding the user;

a subjective risk value acquiring step of acquiring a subjective risk value regarding the user; and

a presenting step of presenting the information regarding the risk to the user based on the subjective risk value and the objective risk value. (Item 7)

An information presentation method executed by an information presentation device that presents information regarding an activity to be taken with respect to a risk pertaining to health of a user, the information presentation method including:

an objective risk value acquiring step of acquiring objective risk values that indicate an objective health-related risk regarding a plurality of users;

a subjective risk value acquiring step of acquiring subjective risk values regarding the plurality of users, the subjective risk values indicating a risk subjectively recognized by the plurality of users;

an activity log acquiring step of acquiring activity logs regarding the plurality of users; and

a presenting step of, for each of the plurality of users, presenting information regarding an activity to be taken with respect to a risk, based on a risk discrepancy value indicating a discrepancy between the subjective risk value and the objective risk value regarding the user and based on the activity log regarding the user.

(Item 8)

A program for causing a computer to function as the units of the information presentation device according to any one of items 1 to 5.

Although embodiments have been described above, the present invention is not limited to these specific embodiments, and various modifications and changes can be made within the scope of the gist of the present invention described in the claims.

### REFERENCE SIGNS LIST

1 Subjective evaluation value storage unit
2 Objective evaluation value storage unit
3 Normalization unit
4 Health risk discrepancy value quantification unit
5 Health risk discrepancy value storage unit
6 Activity log storage unit
7 Normalization unit
8 Variable selection unit
9 Selected variable storage unit
10 Effect measurement unit
11 Result display unit
21 Objective risk value acquisition unit
22 Subjective risk value acquisition unit
23 Presentation control unit
24 Presentation unit
100 Activity estimation device
200 Information presentation device
1000 Drive device
1001 Recording medium
1002 Auxiliary storage device
1003 Memory device
1004 CPU
1005 Interface device
1006 Display device
1007 Input device

1. An information presentation device that presents information regarding a risk to a user, comprising:
a processor; and
a memory storing program instructions that cause the processor to:
acquire an objective risk value regarding the user;
acquire a subjective risk value regarding the user; and
present the information regarding the risk to the user based on the subjective risk value and the objective risk value.

2. The information presentation device according to claim 1,
wherein the risk is a physical risk regarding the user or a mental risk regarding the user.

3. The information presentation device according to claim 1,
wherein the program instructions cause the processor to present the information regarding the risk to the user only if a difference between the objective risk value and the subjective risk value is greater than a predetermined threshold value.

4. An information presentation device that presents information regarding an activity to be taken with respect to a risk pertaining to health of a user, the information presentation device comprising:
a processor; and
a memory storing program instructions that cause the processor to:
acquire objective risk values that indicate an objective health-related risk regarding a plurality of users;
acquire subjective risk values regarding the plurality of users, the subjective risk values indicating a risk subjectively recognized by the plurality of users;
acquire activity logs regarding the plurality of users; and
for each of the plurality of users, present information regarding an activity to be taken with respect to a risk, based on a risk discrepancy value indicating a discrepancy between the subjective risk value and the objective risk value regarding the user and based on the activity log regarding the user.

5. The information presentation device according to claim 4,
wherein the program instructions cause the processor to specify activity logs that are dominant with respect to the risk discrepancy value by performing regression analysis on the risk discrepancy value and the activity log, and to present a name of the activity log as the information regarding an activity to be taken with respect to the risk.

6. An information presentation method executed by an information presentation device that presents information regarding a risk to a user, the information presentation method comprising:
acquiring an objective risk value regarding the user;
acquiring a subjective risk value regarding the user; and
regarding the risk to the user based on the subjective risk value and the objective risk value.

7. (canceled)

8. A non-transitory computer-readable storage medium that stores therein a program for causing a computer to function as the information presentation device according to claim 1.
